(19) United States
(12) Patent Application Publication
(10) Pub. No.: US 2015/0147368 A1
(43) Pub. Date: May 28, 2015

(54) ORAL MOUTH RINSE

(71) Applicant: Bradley Goode, Topanga, CA (US)

(72) Inventor: Bradley Goode, Topanga, CA (US)

(21) Appl. No.: 14/555,181

(22) Filed: Nov. 26, 2014

Related U.S. Application Data
(60) Provisional application No. 61/909,984, filed on Nov. 27, 2013.

(51) Int. Cl. A61K 8/25; A61K 8/02; A61Q 11/00; A61K 8/23

(52) U.S. Cl. CPC A61K 8/25; A61K 8/23; A61K 8/0245; A61Q 11/00; A61K 2800/28
USPC 424/401; 424/49

(57) ABSTRACT

An alkaline intraoral mouth rinse containing a hydrate dissolved in neutral pH distilled water and adjusted to have an alkaline pH, and further including xylitol to complement the alkaline pH to promote chemical/biological disruption of biofilm deposits.

### CROSS REFERENCES TO RELATED APPLICATIONS

The present application is a United States Utility Patent Application and claims the benefit of the filing date of U.S. Provisional Patent Application Ser. No. 61/909,984 filed Nov. 27, 2013 (Nov. 27, 2013), which application is incorporated in its entirety by reference herein.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION BY REFERENCE OF MATERIAL SUBMITTED ON A COMPACT DISC

Not applicable.

### BACKGROUND OF THE INVENTION

1 Field of the Invention

The instant invention relates generally to methods of improving oral hygiene and preventing dental caries, and more particularly to an intra-oral rinse containing hydrated silica suspended in an alkaline water preparation of distilled water, or magnesium sulfate in solution, which, when regularly used as a mouthwash disrupts and aids in eliminating biofilm deposits on teeth and gums.

2. Background Discussion

Hydrated silica is a mild abrasive used to remove debris and residual surface stains in or with such compositions as calcium carbonate, dehydrated silica gels, hydrated aluminum oxides, magnesium carbonate, phosphate salts and silicates. Hydrated silica has been used as an ingredient in food products and is recognized by the U.S. Food & Drug Administration to be Generally Recognized as Safe (“GRAS”) and as having no known carcinogenicity or toxicity. All supplied hydrated silica will be tested by X-ray diffraction guaranty 100% hydrated is used in EOS.

A 2007 study from the Journal of Oral Science found that xylitol-enhanced fluoride toothpaste may help protect against tooth decay. Researchers discovered that the toothpaste helped restore minerals to the teeth (an effect that could hinder the development of cavities).

### BRIEF SUMMARY OF THE INVENTION

An alkaline intraoral mouth rinse containing a hydrate dissolved in neutral pH distilled water and adjusted to have an alkaline pH, and further including xylitol to complement the alkaline pH to promote chemical/biological disruption of biofilm deposits.

### DETAILED DESCRIPTION OF THE INVENTION

The present invention is an alkaline intra-oral mouth rinse containing hydrated silica or magnesium sulfate particles suspended in distilled water. When hydrated silica particles are employed, the average particle diameter is approximately 10 to 25 microns with an approximate concentration of hydrated silica of between 12 and 20 percent (%) by weight. The hydrated silica is suspended in an alkaline preparation of distilled water to a pH 8.1 to 8.5.

The purpose of the hydrated silica is to mechanically disrupt biofilm; the purpose of the alkaline pH solution is to chemically and biologically disrupt biofilm. The rinse washes away the disrupted biofilm and non-calcified plaque from tooth surfaces, as well as crowns and bridges and other fixed dental prostheses. Rinsing with the hydrated silica solution should last for 30 to 60 seconds and then the solution should be fully expectorated.

Xytitol is preferably used as a flavoring agent, as it is known to promote tooth enamel re-mineralization. Ionic calcium and phosphorus may also be used to augment the biofilm disruption and to facilitate washing of the oral environment, as they are integral to tooth enamel re-mineralization.

An alternative compound of magnesium sulfate (“MgSO4.7H2O”) may be used instead of hydrated silica. When added to neutral pH water in a concentration high enough to create a super-saturated solution, the solution allows particles of varying sizes to mechanically disrupt the biofilm and non-calcified plaque. By its nature, the solution is alkaline, and therefore biologically disrupts the plaque.

Magnesium sulfate is a compound with many properties and uses. It is a type of salt as well as a mineral, widely found in the earth's crust. It is obtained from the ore epsomite, a white solid substance found on the surface of the earth. Additionally, it is also found in oceans and fresh water springs. This amazing chemical compound formed by magnesium and sulfate is represented by the molecular formula MgSO₄. Commercially, it is produced by dissolving magnesium carbonate in hot, dilute sulfuric acid. The product thus obtained is magnesium sulfate heptahydrate (the prefix “hepta” referring to the seven molecules of water loosely bound to each of the magnesium sulfate molecules). Magnesium sulfate heptahydrate (“MgSO₄7H₂O”) is commonly known as epsom salt.

When Epsom salt is dissolved in water the resultant pH of the aqueous solution is approximately 6.0, so it is necessary to adjust the pH of the water to arrive at the desired pH of the mouth rinse. A proper stoichiometry accurately determines all concentrations.

Magnesium sulfate injection, USP (50%) for IV and IM use is a sterile concentrated solution of magnesium sulfate USP. Each mL contains magnesium sulfate (magnesium sulfate (magnesium sulfate injection) injection) (heptahydrate) 500 mg, which provides 4.06 mEq each of magnesium (Mg⁺²) and sulfate (SO4⁻²), and water for injection q.s. The pH (5.5-7.0) is adjusted with an acid, such as sulfuric acid, and/or an alkaline solution, such as sodium hydroxide. The solution contains no bacteriostatic agent or other preservatives. The molecular formula is MgSO4•7H2O and the molecular weight is 246.47.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact composition described. Various modifications of the inventive composition, changes in relative amounts of the active and inactive ingredients, as well as equivalents will readily occur to those skilled in the art and may be employed, as suitable, without departing from the true spirit and scope of the invention. Therefore, the above description should not be construed as limiting the scope of the invention, which is defined by the appended claims.

What is claimed as invention is:

1. An alkaline intra-oral mouth rinse solution comprising hydrated silica suspended in distilled water.

2. The mouth rinse of claim 1, wherein said solution has a pH between 8.1 and 8.5, inclusive.

3. The mouth rinse of claim 1, wherein the particle size of said hydrated silica is approximately between 10 to 25 microns, inclusive.

4. The mouth rinse of claim 1, wherein the concentration of said hydrated silica is between 12 and 20 percent by weight.

5. The mouth rinse of claim 1, further including a sugar alcohol.

6. The mouth rinse of claim 5, wherein said sugar alcohol is xylitol.

7. The mouth rinse of claim 1, further including ionic calcium.

8. The mouth rinse of claim 1, further including phosphorus.

9. The mouth rinse of claim 1, further including ionic calcium and phosphorous.

10. An alkaline mouth rinse solution comprising magnesium sulfate dissolved in neutral pH water in a super-saturated concentration.

11. The mouth rinse of claim 10, wherein said magnesium sulfate particle sizes are varied so as to mechanically disrupt biofilm accumulations and non-calcified plaque in a user.

12. The mouth rinse of claim 10, wherein said solution is alkaline.

13. The mouth rinse of claim 10, wherein said magnesium sulfate is of the form having the molecular formula MgSO₄.

14. The mouth rinse of claim 13, wherein said solution is magnesium sulfate heptahydrate having the molecular formula MgSO₄7H₂O.

15. The mouth rinse of claim 14, wherein the pH of said solution is adjusted through the addition of an acid.

16. The mouth rinse of claim 15, wherein said acid is sulfuric acid.

17. The mouth rinse of claim 14, wherein the pH of said solution is adjusted through the addition of a base.

18. The mouth rinse of claim 14, wherein said base is sodium hydroxide.

19. An alkaline intraoral mouth rinse containing a hydrate dissolved in neutral pH distilled water and adjusted to have an alkaline pH.

20. The mouth rinse of claim 19, further including xylitol.
